(19) United States
(12) Patent Application Publication
(10) Pub. No.: US 2009/0281364 A1
(43) Pub. Date: Nov. 12, 2009

(54) Metathesis process using a moving phase reactor

(76) Inventor: Richard B. Halsey, Houston, TX (US)

Correspondence Address: LyondellBasell Industries, 3801 WEST CHESTER PIKE, NEWTOWN SQUARE, PA, 19073, US

(21) Appl. No.: 12/152,053

(22) Filed: May 12, 2008

(51) Int. Cl. C07C 6/04

(52) U.S. Cl. 585/646

(57) ABSTRACT

A method for metathesizing at least two gaseous olefins using a moving catalyst bed gas phase metathesis reactor and counter current flowing subdivided solid catalyst.

### BACKGROUND OF THE INVENTION

1. Field of the Invention

This invention relates to the metathesis (disproportionation) of olefins. More particularly, it relates to a process for carrying out a metathesis reaction in a moving catalyst bed, gas phase reactor using a subdivided, solid catalyst that promotes the metathesis reaction.

2. Description of the Prior Art

The catalyzed metathesis of olefins was first disclosed in 1964, and, because of its versatility, has since developed into a whole new field of its own within the universe of hydrocarbon chemistry.

Basically, the metathesis process utilizes a double bond displacement mechanism that involves the breaking and reformation of olefinic bonds, the type and number of bonds remaining unchanged. Starting with two different olefinic molecules, the reaction causes the displacement of double bond groups from each molecule to produce two new olefinic molecules that are not the same as the starting molecules. Displacement cleavage occurs at a double bond on each starting olefin molecule, and different olefin molecules are formed that have double bonds where the old double bonds were cleaved. For example, propylene is currently commercially produced by metathesizing 2-butene with an excess of ethylene. In this particular process, the double bonds in a molecule of 2-butene are cleaved as are the double bonds in a molecule of ethylene, and the resulting radicals reform to produce two new molecules of propylene. The process can be promoted with either homogeneous or heterogeneous catalyst systems comprised of one or more functional catalysts.

The metathesis of olefins is well understood and is fully and completely disclosed in U.S. Pat. No. 6,872,862 to Bridges, Powers, and Coleman.

Heretofore, metathesis reactions such as the propylene production process discussed above have been carried out using a fixed bed of catalyst through which flows the fluid (gas and/or liquid) olefin reactants, see U.S. Pat. Nos. 5,026,936 and 6,872,862. The catalyst employed in these fixed beds is a solid particle, typically pellet size, e.g., about 1/16 to ¼ inch in diameter and about 1/16 to ¼ inch in length.

Metathesis reactor (reactor) cycles between catalyst regeneration operations are often dictated by the pressure drop across the reactor. For example, the pressure drop across a reactor can climb steadily over the course of 2 to 4 weeks from an initial pressure of about 2 to 10 psig to a final pressure of over 30 psig. At this point in time in the operation of the reactor, the catalyst bed is sufficiently fouled to require shutdown of the process and a catalyst regeneration operation.

This pressure drop is usually caused by catalyst pellet attrition resulting in a buildup of catalyst fines in the reactor, or coke deposition on the catalyst pellets, or both. As the catalyst ages, accumulated catalyst fines in the catalyst bed increase not only the initial pressure drop across the bed, but also the rate of increase of the pressure drop over the period of time the bed is in operation.

Accordingly, it is desirable to have a metathesis process that is not subject to the vagaries of catalyst attrition and coke deposition in a fixed catalyst bed.

### SUMMARY OF THE INVENTION

Pursuant to this invention a metathesis process is provided that employs a moving catalyst bed/gas phase reactant metathesis reactor, and counter current flow between the solid catalyst and the vaporous reactants in that reactor.

### BRIEF DESCRIPTION OF THE DRAWINGS

FIG. 1 shows a simplified flow sheet of a prior art metathesis process using a fixed bed of catalyst.

FIG. 2 shows a flow sheet of one embodiment within the process of this invention using a moving catalyst bed reactor.

### DETAILED DESCRIPTION OF THE INVENTION

For sake of clarity and brevity, this invention will be described in respect of the metathesis of 2-butene with ethylene to form propylene, but this invention is not so limited in its scope.

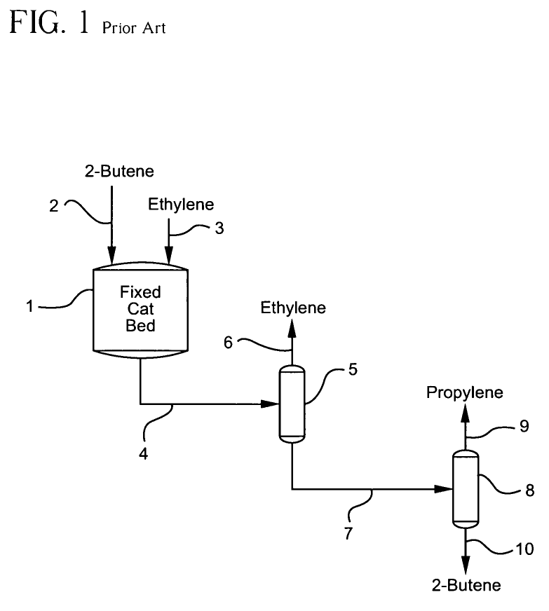

FIG. 1 shows a fixed bed of catalyst 1 into which flows reactant stream 2 composed of 2-butene and reactant stream 3 composed of ethylene. Catalyst bed 1 is maintained at operating conditions that favor, in the presence of the catalyst, the cleavage of double bonds in both the ethylene and 2-butene and the reformation of the resulting radicals into the desired propylene product.

The reaction mixture containing unreacted ethylene and 2-butene feeds and propylene product is passed by way of line 4 to a distillation column 5 that separates ethylene 6 as overhead from the reaction mixture for recycle to bed 1, if desired.

Bottoms stream 7 of column 5 is composed primarily of 2-butene and propylene. This mixture is separated into propylene product stream 9 and separate bottoms stream 10. Stream 10, composed essentially of 2-butene, can also be recycled to bed 1, if desired.

It is in bed 1 that attrited catalyst fines and/or coke can collect and drive the pressure drop across bed 1 (from inlets 2 and 3 to outlet 4) up to a level that requires the metathesis process to be terminated, and the catalyst in bed 1 regenerated or replaced.

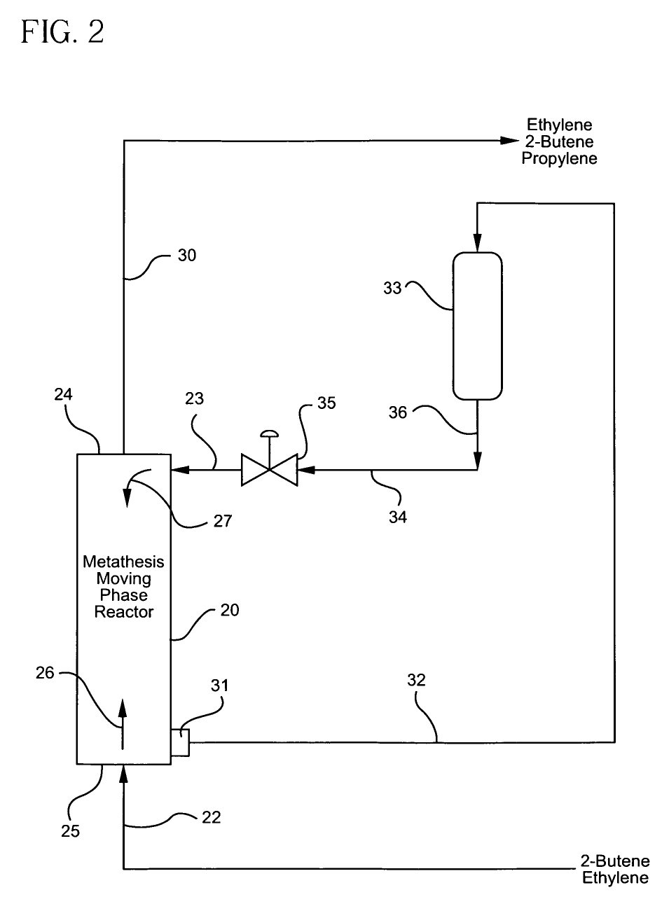

FIG. 2 shows one flow scheme within this invention. In this Figure a moving catalyst bed reactor 20 receives by way of conduit 22 a gaseous mixture of ethylene and 2-butene reactants, and by way of conduit 23 subdivided, solid metathesis promoting catalyst.

In this embodiment, reactor 20 has upper and lower opposed ends 24 and 25, respectively. Vaporous reactants 22 enter at or near bottom end 25, and, by force of a pressure gradient across the height of reactor 20, flow upwardly inside reactor 25, as shown by arrow 26, toward top end 24. Solid catalyst particles enter at or near upper end 24, and, by force of gravity, flow downwardly, as shown by arrow 27, into counter current flow contact and mixing with rising reactants 22, thereby promoting the desired metathesis reaction. In this particular embodiment, reactor 20 is maintained at operating conditions that favor the conversion of one mole of ethylene and one mole of 2-butene to two moles of propylene.

The mixture of propylene product and unreacted ethylene and 2-butene is removed by way of line 30, and passed elsewhere for further processing to separate the propylene product from the unreacted ethylene and 2-butene. The thus recovered unused reactants can be returned by way of line 22 to reactor 20 as feed therefore. For example, the reaction mixture in line 30 can be processed in columns 5 and 8 of FIG. 1, to recover the desired propylene product and the recycle feed reactants.

Solid catalyst particles that reach the bottom of reactor 20 are collected in a conventional solid/gas separator 31 so that essentially only solid catalyst passes from the interior of the lower end of reactor 20 into a mechanical catalyst conveyor 32 that conveys the degassed solid catalyst to a conventional regeneration unit 33. In unit 33, coke can be air burned in a conventional manner, and thereby removed from the catalyst particles. The regenerated catalyst is then gravity fed into line 34, through catalyst lock out valve 35. Valve 35 is normally maintained at least partly open for the passage of catalyst there through. From valve 35 the catalyst passes back into reactor 20 by way of conduit 23 to promote additional metathesis reaction.

The process of FIG. 2 addresses the problems of catalyst attrition and/or catalyst coking causing unacceptable pressure drops across the metathesis reactor, thereby allowing reactor 20 to operate continuously, and a substantially longer time, even years longer, between reactor shutdowns.

The process of this invention also allows for almost infinite flexibility for varying the make-up of the reactant/catalyst mixture that is to be subjected to metathesis conditions in reactor 20. For example, a 2-butene reactant stream may not be wholly 2-butene. It may contain minor amounts of 1-butene, and the amount of 1-butene contained in a reactant stream can vary over time. Reactant stream compositions change over time of operation, e.g., the 1-butene content in a 2-butene stream can vary. By the process of this invention, the amount of ethylene and/or catalyst mixed with 2-butene feed component can be changed to accommodate the varying amount of 1-butene present in that component feed. For example, if the 2-butene reactant contains varying amounts of 1-butene, and one of the catalyst components has olefin isomerization functionality (i.e., magnesium oxide), the magnesium oxide level in the catalyst passed to reactor 20 can be increased in conduit 34 by way of line 36, in any amount desired to isomerize at least part of the increasing 1-butene content in the feed. Similarly, if the 1-butene content decreases, a matching decrease in magnesium oxide content can, with this invention, easily be affected by removal of catalyst from unit 33 in a conventional manner well known in the art. Thus, by this invention superior flexibility in operation is possible since the catalyst composition can be tailored to meet varying compositions of the reactant mix 22, and thereby carry out a more efficient process.

The two or more reactants that form mix 22 can vary widely so long as they are olefins, with alpha or internal un-saturation. Generally, they can be monoolefins having from 2 to 8 carbon atoms per molecule (C2 to C8 olefins).

Suitable metathesis promoting catalysts include at least one of halides, oxides and/or carbonyls of molybdenum, tungsten, rhenium, and/or magnesium carried on a support, preferably an oxide support such as silica, alumina, titania, zirconia and mixtures thereof. Activating agents can also be included in the catalyst make-up. Such agents can include organo-metallic compounds such as tetra methyl tin; oxides such as alkaline earth metal oxides, alumina, silica, and mixtures thereof.

Pursuant to this invention the catalyst or catalyst combinations employed can vary widely in its subdivided form. The solid particle range for the catalyst mixture can vary from powdered catalyst of from about 0.1 inch up to right cylindrical pellets of catalyst having lengths up to about 1 inch and diameters up to about one-half inch, and any combination of particle sized in between so long as they can be made to flow counter currently with the gaseous feed reactants rising in reactor 20.

The operating conditions maintained in reactor 20 can vary widely, but will generally be a temperature of from about 300 to about 800 degrees Fahrenheit (F), and a pressure of from about 200 to about 600 psig. The pressure differential maintained over the length of reactor 20 can vary widely depending on the particle size make-up and distribution, but will in all cases be a differential within reactor 20 that is sufficient to maintain essentially continuous flow of reactant feed through the reactor against the counter currently flowing solid catalyst load. For example, the pressure differential can vary from about 600 psig at the feed inlet end of the reactor and about 200 psig at the outlet end of the reactor, or any differential within that 200 to 600 psig pressure range.

Reactor 20 can be a conventional counter current flow reactor known in the art. In the operation of counter current flow reactors pursuant to this invention, solid, particulate catalyst particles as defined above are made to flow into, and mix with, counter flowing pressurized feed mixture 22. This mixing process causes intimate contact of reactants and catalyst in reactor 20. Metathesis occurs while the mixture of reactants and catalyst pass by one another in the interior of reactor 20, and, at the same time, are subjected to metathesis favoring operating conditions.

For example, in a moving bed reactor 20 system such as that shown in FIG. 2, the catalyst particles gravity flow downwardly inside the open interior of the reactor relative to the reactor wall, and typically maintain their positions relative to one another as they flow downwardly. Plug flow of both the catalyst and the reactants through the reactor is readily achievable and desirable.

With the moving bed system of this invention catalyst can be withdrawn from the process either continuously or intermittently or any desired combination thereof. Thus, catalyst can be regenerated outside the system, replaced, and/or reintroduced into the system at will.

Catalyst can, for example, be removed from the reactor by gravity into a conventional standpipe (not shown) that is located below the point where the reactants are introduced into the lower part of reactor 20. The standpipe can contain a typical interlock valve system (not shown) that cycles between open and closed positions with a side vent that isolates the catalyst removed from the reactor while the reactor is in operation. Once the catalyst is below the isolating valve system, the catalyst is moved downwardly into a receiving hopper (not shown) that employs a mechanical conveyor or flowing motive gas system, represented by line 32, to transport the spent catalyst upward to regenerator 33. After catalyst fines removal, if necessary, the regenerated catalyst is moved by gravity into an interlock system represented by valve 35 for reintroduction into the upper part of the reactor for reuse in the process.

As stated above, the reaction conditions can vary widely depending on the particular reactants and catalyst system used, so the physical configuration and orientation of the reactor can also vary widely. However, reactor 20, as shown in the embodiment of FIG. 2 will generally have a vertical height of from about 1 to about 100 feet thereby providing a reactant 22 residence time of from about 10 milliseconds to about 10 minutes inside the reactor itself in contact with the catalyst.

### EXAMPLE

A gaseous mixture of about 16 weight percent (wt %) 1-butene and about 84 wt % 2-butene together with a molar excess ethylene is passed into the bottom of reactor 20 at a pressure of about 350 psig. The pressure at the top of reactor 20 is about 330 psig so that the vaporous reactant mixture rises toward the top of reactor 20. Catalyst pellets composed of tungsten oxide and magnesium oxide, and about one-half inch long and about one-eighth inch in diameter are employed in conduit 23. The counter current flowing mixture of reactants and catalyst inside reactor 20 is maintained at a temperature of about 600 F Reactor 20 is operated at a reactant feed flow rate that provides a residence time for the reactants in the reactor of about 10 minutes.

A mixture of propylene, unreacted ethylene, unreacted 2-butene, 1-butene, and propylene is recovered overhead from the reactor, and the propylene separated there from as a product of the process.

I claim:

1. In a method for metathesizing at least two gaseous olefin reactants wherein said olefin reactants are contacted with a metathesis promoting catalyst under operating conditions that favor the transformation of said olefin reactants to at least one other olefin as the product of the process, the improvement comprising carrying out said metathesis using subdivided solid metathesis catalyst in a moving catalyst bed gas phase reactor that has opposing ends, said at least two gaseous olefin reactants being introduced into said reactor near one of said opposing reactor ends, said solid metathesis catalyst being introduced into said reactor near the other of said opposing reactor ends, and passing said olefin reactants into counter current contact with said metathesis catalyst in said reactor under metathesis promoting conditions of operation.

2. The method of claim 1 wherein said olefins are selected from the group consisting of C2 to C8 olefins having at least one of alpha or internal double bonds.

3. The method of claim 1 wherein said subdivided solid metathesis catalyst has a particle size of from about 0.1 inch up to right cylindrical pellets of up to about one inch in length and up to one half inch in diameter.

4. The method of claim 1 wherein said catalyst is at least one of halides, oxides, and carbonyls of at least one of molybdenum, tungsten, rhenium, and magnesium carried on a solid support.

5. The method of claim 4 wherein said catalyst contains at least one activating agent.

6. The method of claim 1 wherein said operating conditions are a temperature of from about 300 to about 800 F, and a pressure of from about 200 to about 600 psig.

7. The method of claim 1 wherein said olefin reactants are at least ethylene and 2-butene, said catalyst is magnesium oxide and tungsten oxide supported on silica, and said at least one other olefin product is propylene.

8. The method of claim 1 wherein said solid metathesis catalyst is passed downwardly in said reactor and said gaseous olefin reactants are passed upwardly in said reactor, whereby said catalyst and said reactants contact one another and mix with one another in counter current fashion.
